SEQUENCE LISTING (16 sequences)

SEQ ID NO: 1 - DNA, 2215 bp, Oryza sativa
  CDS (63)..(1949): DNA sequence encoding OXHS4
gagagagagacgacgtgctcatcgatctggttttggttggaaggaggcgcttcaggtctgccatggatttcacctccgacgagtcagagctaagtgatgcagatattgatgactatgctgacaagtgctacatggacctcaagtctgggaagccggtggtgagtctaggcaatgaaaagttcagatgcccattctgtctggggaagaagaagcaggactaccgttacaacgagctacttcagcatgccattggggttggcgcatccaatcgcgctccaaaggtgaaggcaaaccacatggccttggccaatcttctcaagaacgactatgctgatgcagcaggctcattgccatcacgacaggccattggaccaagtaatcctccaaggccattgcaagatcaggaagcgtatgtttggccatggatgggcatccttgcaaatgttccagctgagaaaacaaaggaggatggagctagtctgatgcagcagctagctaatttcaatcccttgcagtttactgctgtgctctgctccgagggtaggtatactggttatgcagttgtcggtttcagcaaagattggattgggttcacgaacgccttggcattccacaactacttcaaatcacaacgtctgggtaaaaaggattgggcagcacttggacaagagaagtacatctgtggatggatggcaaaagaagaggactacaaatctagtgacccagttggtaggtttttgtcagcaaatggtgatctgaagaccgtgtctggtctggaaaatgacctgtctcgcaagactgagactctcatagctaacttgtcacaccagattacagctaagagcaagtatttggtggaactggaatgcagatgcaatcaaatgaatctctctgttaaaagggcaatggaagaaactgacttgctgcacaaaagttacaatgaagaaatgcgaaatatgcaatctgctgctcgtgaacattcacagaaaatatttgaagagactgatcagctgaggaaacagttggatgataaagagaatgccatcgagaggagatccaagcaactaagcaagtttgttgctcaaactgacatagaaagaagaaaattggagagtgagatgaaaaagaactctgagcaaaacgactccatccacatggctagaatcgagcaacagaagtctgataaaaatgtgctaaagcttgttgagaaacacaagaaagagaaggagattgctctgaacaaaatcctgcagttagaaaagcaattggatgaaaagcaaaagctggaactggaaatacaacaacttagaggaagactgctggtggtccaacacatggagggagagggtgttgatgtgaagaaacgtactgacgagctgactgaggaactaaatgagaagattgaggagatggaatatatggaaggtctgaatcaaactctcattatcaaagaaaggaagacaaatgatgaactgcaagatgccaagaaagcgctgatttctggcttatcagaattgttaggccctcgtagcaccatcggactcaagagaatgggcgagctggatgagaaacccttccttgcagcttgcaagaaaaggtatggtactgctgacggtgaagctgaaataaaagctgctgaattttgctccgagtggcaagaaaatctgaaggatgccaattggcatccgttcaagattgttaccagaggaggaaagactgagcaaatcatcaatgaagatgatgagaagctagtgggcttgaaggagcagctcggtgacgaggtctacaaggccgtgaccacggcgctgctggagatcaacgagtacaatgccagcggcagctacgtggtgtcagagctgtggaacaacaaggaggacaggaaggccagcatgcaggaggccttgcagcacgtcctggagcagtggaagcttcggaggcgaaggagatgaagcacgcctttcactcacactggattgaactaaggctttttgttcatgcttcgtagtgctcagttattttttggatgggattatcttggcttagctgaaactggaagattatgcattgtgtgatcctggtttctcgctctgattgaaatcgctgggataaacacattaaggtcatgaactacattctgatagaattaactttctgatattcactacattctgatagaattgtgctgtcagagcataaatctttttgggacagtaat

SEQ ID NO: 2 - protein, 628 aa, Oryza sativa
  amino acid sequence of OXHS4
MDFTSDESELSDADIDDYADKCYMDLKSGKPVVSLGNEKFRCPFCLGKKKQDYRYNELLQHAIGVGASNRAPKVKANHMALANLLKNDYADAAGSLPSRQAIGPSNPPRPLQDQEAYVWPWMGILANVPAEKTKEDGASLMQQLANFNPLQFTAVLCSEGRYTGYAVVGFSKDWIGFTNALAFHNYFKSQRLGKKDWAALGQEKYICGWMAKEEDYKSSDPVGRFLSANGDLKTVSGLENDLSRKTETLIANLSHQITAKSKYLVELECRCNQMNLSVKRAMEETDLLHKSYNEEMRNMQSAAREHSQKIFEETDQLRKQLDDKENAIERRSKQLSKFVAQTDIERRKLESEMKKNSEQNDSIHMARIEQQKSDKNVLKLVEKHKKEKEIALNKILQLEKQLDEKQKLELEIQQLRGRLLVVQHMEGEGVDVKKRTDELTEELNEKIEEMEYMEGLNQTLIIKERKTNDELQDAKKALISGLSELLGPRSTIGLKRMGELDEKPFLAACKKRYGTADGEAEIKAAEFCSEWQENLKDANWHPFKIVTRGGKTEQIINEDDEKLVGLKEQLGDEVYKAVTTALLEINEYNASGSYVVSELWNNKEDRKASMQEALQHVLEQWKLRRRRR

SEQ ID NO: 3 - DNA, 21 bp, Artificial Sequence
  OXHS4-1F primer for amplifiying OXHS4 gene
tgacgaggtctacaaggccgt

SEQ ID NO: 4 - DNA, 19 bp, Artificial Sequence
  OXHS4-1R primer for amplifying OXHS4 gene
acaccacgtagctgccgct

SEQ ID NO: 5 - DNA, 21 bp, Artificial Sequence
  AF primer for amplifying a 76 bp fragment of the rice Actin1 gene (Accession No. X16280)
tggcatctctcagcacattcc

SEQ ID NO: 6 - DNA, 20 bp, Artificial Sequence
  AR primer for amplifying a 76 bp fragment of the rice Actin1 gene (Accession No. X16280)
tgcacaatggatgggtcaga

SEQ ID NO: 7 - DNA, 18 bp, Artificial Sequence
  OXHS4OXF-sequence specific primer with one KpnI cleavage site
aggaggcgcttcaggtct

SEQ ID NO: 8 - DNA, 20 bp, Artificial Sequence
  OXHS4OXR-sequence specific primer with one BamHI cleavage site
gcataatcttccagtttcag

SEQ ID NO: 9 - DNA, 560 bp, Artificial Sequence
  sequence flanking the OXHS4 T-DNA mutant 1C-03064
atcaactcacctggtacctggtacctcggatccgtgacctcaagctctgggaanccaggtggatgagtcgtaggcaatgaaaagttcanatgcccattctgtcatgggtgaagtaagaatgcaggactaccgttncaacnagctacttcancatgccattggggtatggcgcatccaatcgctctccaaaggtgaaggcanaccanntggncttggccantcttctcaagaacnactatgctgatgcancaggctcattgccatcacgacaggccattggaccaagtaatcctccaaggccattgcaagatcaggaagcgtatgtttggccatggatgggcatccttgcaaatgttccagctgagaaaacaaaggaggatgganctagtctgatgcancagctanctaatttcaatcccttgcagtttactgctgcgctctgctcccaggtaggtatactggttatgcagttgtccgtttcncananatnggattgggttcacgaacnccttgnnnttncanaactncttnaaatcnnacgtctggnnanaaagattgcc

SEQ ID NO: 10 - DNA, 20 bp, Artificial Sequence
  primer A flanking the insertion site
tgaacacacatcagtgagtt

SEQ ID NO: 11 - DNA, 20 bp, Artificial Sequence
  primer B flanking the insertion site
caccaaatacttgctcttag

SEQ ID NO: 12 - DNA, 23 bp, Artificial Sequence
  primer T at the boundary of vector
ttggggtttctacaggacgtaac

SEQ ID NO: 13 - DNA, 19 bp, Artificial Sequence
  OXHS4RTF primer
ccgtgtctggtctggaaaa

SEQ ID NO: 14 - DNA, 19 bp, Artificial Sequence
  OXHS4RTR primer
tcttgagtccgatggtgct

SEQ ID NO: 15 - DNA, 23 bp, Artificial Sequence
  OXHS4QF primer
gtgacccagttggtaggtttttg

SEQ ID NO: 16 - DNA, 21 bp, Artificial Sequence
  OXHS4QR primer
ttgcgagacaggtcattttcc

(19) United States
(12) Patent Application Publication
(10) Pub. No.: US 2015/0218581 A1
(43) Pub. Date: Aug. 6, 2015

(54) Use of OXHS4 Gene in Controlling Rice Drought Resistance

(71) Applicants: Lizhong Xiong, Wuhan (CN); Yonghua Tan, Wuhan (CN)

(72) Inventors: Lizhong Xiong, Wuhan (CN); Yonghua Tan, Wuhan (CN)

(73) Assignee: HUAZHONG AGRICULTURAL UNIVERSITY, Wuhan (CN)

(21) Appl. No.: 14/111,633

(22) PCT Filed: Apr. 16, 2012
(86) PCT No.: PCT/CN12/74106

§ 371 Date: May 19, 2014

(30) Foreign Application Priority Data
Apr. 15, 2011 (CN) 201110094033.3

(51) Int. Cl. C12N 15/82; A01G 9/00

(52) U.S. Cl. CPC C12N 15/8273; A01G 9/00

(57) ABSTRACT

Rice OXHS4 gene controlling root growth and conferring enhanced drought resistance, and use thereof in genetic improvement of rice drought resistance is provided. OXHS4 gene is cloned using candidate gene screening method. Drought stress experiments at seedling stage and adult plant stage shows that overexpression of OXHS4 gene can improve drought resistance of transgenic rice, while the oxhs4 mutant exhibits sensitivity to drought, showing the function of said gene and the use thereof.

### TECHNICAL FIELD

The present invention relates to the field of rice genetic engineering. In particular, the present invention relates to obtaining rice OXHS4 gene controlling root growth and conferring enhanced drought resistance by isolation, cloning and function verification and to the use of the gene in genetic improvement of rice drought resistance. In the present invention, the applicant cloned OXHS4 gene controlling rice drought resistance using candidate gene screening method. Drought stress experiments at seedling stage and adult plant stage shows that overexpression of OXHS4 gene can improve the drought resistance of transgenic rice, while an oxhs4 mutant exhibit sensitive to drought, showing the function of said gene and the use thereof.

### BACKGROUND ART

As the main food crop in China and even worldwide, yield and quality of rice is seriously limited by abiotic stress such as drought, salt and coldness, which greatly influences the agricultural production and even social life. It makes great sense to exploit novel genes related to stress and deeply understand stress resistant mechanisms thereof for enhancing rice stress resistance and improving rice variety. Root system is the critical organ of plant responsible for directly absorbing water and has a crucial effect on the drought resistant function of plant. The growth and improvement of crop root system is beneficial to water absorption under drought conditions and advanced root system is also beneficial for increasing plant survival and recovery capability. Therefore, root system growing in depth and width directions is one of the main strategies for crop to survive under drought conditions. Advanced root system in depth and width directions permits plants to fully absorb water in soil and to survive drought stage. With further understanding the root architecture, it has been found that the growth of Root Cortical Aerenchyma (RCA) directly affects the drought resistant capability of plant. Under drought condition, advanced cortical aerenchyma can reduce root metabolic costs, permitting greater root growth and more water acquisition (Zhu et al., Root cortical aerenchyma improves the drought tolerance of maize (Zea mays L.). Plant Cell Environ. 2010 May; 33(5):740-9). For better understanding the growth mechanisms of root system under drought conditions, in transcriptional level, gene expression was profiled in the root cap and elongation zone of the maize primary root under well-watered and water-stressed conditions. It was found that upon water stress, the expression levels of genes involving different pathways were significantly distinct in spatial dimension, particularly, in root cap, the expression levels of genes belonging to reactive oxygen species and carbon metabolism were mostly different before and after water stress; while in elongation zone, expression of the genes related to cell elongation differed most (Spollen et al., Spatial distribution of transcript changes in the maize primary root elongation zone at low water potential, BMC Plant Biol. 2008 Apr. 3; 8:32.). This suggests that related genes perform the critical functions in their own regions upon plant suffering from water stress, thus pointing out the direction for studying the function of the root region specific expressed genes and the effects of root growth related mutant under drought conditions. With the application of QTL, development of microarray data and large scale screening of mutants, many genes related to root development and also affecting plant drought resistance have been found. In Arabidopsis, active gene HDG11 expressing EDT1 and HDG11 overexpession plant had longer primary roots and more lateral roots compared with wild type plants and the well grown roots system significantly enhanced the drought resistance of the plants (Yu et al., Activated expression of an Arabidopsis HD-START protein confers drought tolerance with improved root system and reduced stomatal density, Plant Cell. 2008 April; 20(4): 1134-51.). With rice OsNAC10 being driven by root specific promoter RCc3, root diameter of the transgenic plants was thicker by 1.25-fold than wild type plants, thereby resulting in 5% to 14% grain yield increase of transgenic plants under normal growth conditions and 25% to 42% grain yield increase of OsNAC10 overexpression plants under drought conditions due to enhanced root system contributing to the increase of drought resistance (Jeong et al., Root-specific expression of OsNAC10 improves drought tolerance and grain yield in rice under field drought conditions, Plant Physiol. 2010 May; 153(1):185-97.). In cotton plants, the overexpression of Arabidopsis thaliana gene AVP1 activates auxin transport in the root system and stimulates the rapid growth of root, which enhances the ability of root to absorb water under drought conditions (Pasapula et al., Expression of an Arabidopsis vacuolar H+-pyrophosphatase gene (AVP1) in cotton improves drought- and salt tolerance and increases fibre yield in the field conditions, Plant Biotechnol J. 2011 January; 9(1):88-99.). The overexpression of xylophyta-derived cyclophilin in Arabidopsis thaliana significantly enhanced the root growth under osmosis stress (Sekhar et al., Isolation and characterization of a pigeonpea cyclophilin (CcCYP) gene, and its over-expression in Arabidopsis confers multiple abiotic stress tolerance, Plant Cell Environ. 2010 Aug. 1; 33(8):1324-38.). AtSQE is responsible for sterol synthesis in root and loss of SQE1 results in the growth deficiency of root and thereby obviously decreased drought resistance (Pose et al., Identification of the Arabidopsis dry2/sqe1-5 mutant reveals a central role for sterols in drought tolerance and regulation of reactive oxygen species, Plant J. 2009 July; 59(1):63-76). Arabidopsis thaliana hrd-D mutant exhibits increased secondary roots and thicker root cortex, thus improving water use efficiency of water absorption and resulting in increased drought resistance (Karaba et al., Improvement of water use efficiency in rice by expression of HARDY, an Arabidopsis drought and salt tolerance gene, Proc Natl Acad Sci USA. 2007 Sep. 25; 104(39):15270-5). From the above, root growth has a critical effect on drought resistance of plants. Meanwhile, regulation of root development by hormones exists not only under normal conditions, but stronger under stress conditions. According to the difference of drought resistant mechanisms, these genes can be classified into two groups: group I is the genes regulating growth and development of root system while being not induced by drought stress; group II is the genes controlling root growth and meanwhile being induced by drought stress. Improvement of the latter genes makes greater sense for enhancing crop drought resistance.

### SUMMARY OF THE INVENTION

One object of the present invention relates to the use of OXHS4 gene, a member of XHS family in controlling drought resistance improvement in a plant such as rice. OXHS4 encodes 628 amino acids and is a typical XHS protein with complete domains comprising Zf-XS, XS, Coiled-Coil and XH domains. The present invention isolated and used a cDNA fragment containing OXHS4 gene, which confers the increased resistance to drought stress in a plant such as rice. The sequence of said cDNA containing OXHS4 gene is set forth in SEQ ID NO:1 with 2215 bp in length, wherein the ORF (coding region) with 1887 bp in length is set forth as position 63-1949 in SEQ ID NO:1, encoding 628 amino acids. The amino acid sequence of the encoded protein is set forth in SEQ ID NO: 2. It has been demonstrated in the present invention that the rice OXHS4 gene of the present invention confers increased resistance to drought stress in plants such as rice.

Therefore, the present invention relates to the following embodiments among others:

Item 1. Use of OXHS4 gene controlling drought resistance in genetically improving drought resistance of a plant, wherein said gene encodes an amino acid sequence as set forth in SEQ ID NO: 2.

Item 2. The use according to Item 1, wherein said gene has a coding sequence as shown from nt position 63 to nt position 1949 of SEQ ID NO. 1.

Item 3. The use according to Item 1, wherein said gene has a nucleotide sequence as set forth in SEQ ID NO. 1.

Item 4. A method of improving drought resistance of a plant, wherein said plant is subjected to a treatment so that OXHS4 gene is expressed at an increased level in said plant in comparison with an identical control plant without the treatment, wherein said gene encodes an amino acid sequence as set forth in SEQ ID NO: 2.

Item 5. The method according to Item 4, wherein said gene has a coding sequence as shown from nt position 63 to nt position 1949 of SEQ ID NO. 1.

Item 6. The method according to Item 4, wherein said gene has a nucleotide sequence as set forth in SEQ ID NO. 1.

Item 7. The method according to any one of Items 4-6, wherein said treatment is transformation of said plant by a DNA construct comprising said gene.

Item 8. A DNA construct comprising OXHS4 gene, wherein said gene encodes an amino acid sequence as set forth in SEQ ID NO: 2.

Item 9. The DNA construct according to Item 8, wherein said gene has a coding sequence as shown from nt position 63 to nt position 1949 of SEQ ID NO. 1.

Item 10. The DNA construct according to Item 8, wherein said gene has a nucleotide sequence as set forth in SEQ ID NO. 1.

Item 11. A transgenic plant or a cell thereof comprising transformed OXHS4 gene in its genome, wherein said gene encodes an amino acid sequence as set forth in SEQ ID NO: 2.

Item 12. The transgenic plant or cell according to Item 11, wherein said gene has a coding sequence as shown from nt position 63 to nt position 1949 of SEQ ID NO. 1.

Item 13. The transgenic plant or cell according to Item 11, wherein said gene has a nucleotide sequence as set forth in SEQ ID NO. 1.

In any one of the above Items 1-13, said plant is selected from the group consisting of corn, cotton, soybean, rice and wheat plants, preferably said plant is rice.

### DETAILED DESCRIPTION OF THE INVENTION

Unless specifically defined, all technical and scientific terms used herein have the same meaning as commonly understood by persons of ordinary skill in the art. The procedures for preparing and screening transgenic plants described below are well known and commonly employed by persons of ordinary skill in the art.

The OXHS4 gene of the present invention is useful in genetically improving drought resistance of a plant such as rice. The gene can be engineered into a vector to form a DNA construct. The DNA construct can be used in transformation of a plant cell or tissue of a plant such as rice to produce a transgenic plant cell and/or transgenic plant. In the transgenic plant cell and transgenic plant, the OXHS4 gene of the present invention is expressed in an increased level which confers increase level of drought resistance. Therefore, the present invention also relates to a method of improving drought resistance of a plant, wherein said plant is subjected to a treatment so that the OXHS4 gene of the present invention is expressed at an increased level in said plant in comparison with an identical control plant without the treatment. The OXHS4 gene, DNA construct comprising said gene, plant cells or plants comprising transformed OXHS4 gene and use and method of use of the gene of the present invention for improving drought resistance of a plant such as rice are all encompassed in the present invention.

A transgenic “plant cell” means a plant cell that is transformed with stably-integrated, non-natural, recombinant polynucleotides, e.g. by Agrobacterium-mediated transformation or by bombardment using micropaiticles coated with recombinant polynucleotides. A plant cell of this invention can be an originally-transformed plant cell that exists as a microorganism or as a progeny plant cell that is regenerated into differentiated tissue, e.g. into a transgenic plant with stably-integrated, non-natural recombinant polynucleotides in its chromosomal DNA, or seed or pollen derived from a progeny transgenic plant.

A “transgenic” plant or seed means one whose genome has been altered by the stable incorporation of recombinant polynucleotides in its chromosomal DNA, e.g. by transformation, by regeneration from a transformed plant from seed or propagule or by breeding with a transformed plant. Thus, transgenic plants include progeny plants of an original plant derived from a transformation process including progeny of breeding transgenic plants with wild type plants or other transgenic plants. The enhancement of a desired trait can be measured by comparing the trait property in a transgenic plant which has recombinant DNA conferring the trait to the trait level in a progenitor plant.

“Gene expression” means the function of a cell to transcribe recombinant DNA to mRNA and translate the mRNA to a protein. For expression the recombinant DNA usually includes regulatory elements including 5′ regulatory elements such as promoters, enhancers, and introns; other elements can include polyadenylation sites, transit peptide DNA, markers and other elements commonly used by those skilled in the art. Promoters can be modulated by proteins such as transcription factors and by intron or enhancer elements linked to the promoter.

“An increased level” of expression means an increase in the gene expression that is helpful for the drought resistance of the plant, e.g., at least about 5%, at least about 10%, at least about 15%, at least about 20%, at least about 25%, at least about 30%, at least about 40% at least about 50%, at least about 100%, at least about 200% increase in comparison with an identical control without the treatment of the present invention.

“Recombinant polynucleotide” means a DNA construct that is made by combination of two otherwise separated segments of DNA, e.g., by chemical synthesis or by the manipulation of isolated segments of nucleic acids by genetic engineering techniques. Recombinant DNA can include exogenous DNA or simply a manipulated native DNA. Recombinant DNA for expressing a protein in a plant is typically provided as an expression cassette which has a promoter that is active in plant cells operably linked to DNA encoding a protein, linked to a 3′ DNA element for providing a polyadenylation site and signal. Useful recombinant DNA also includes expression cassettes for expressing one or more proteins conferring stress tolerance.

Recombinant DNA constructs generally include a 3′ element that typically contains a polyadenylation signal and site. Well-known 3′ elements include those from Agrobacterium tumefaciens genes such as nos 3′, tml 3′, ttnr 3′, tms 3′, ocs 3\ tr73′, e.g., disclosed in U.S. Pat. No. 6,090,627. 3′ elements from plant genes such as a rice glutelin gene, a rice lactate dehydrogenase gene and a rice beta-tubulin gene are disclosed in U.S. published patent application 2002/0192813 A1.

The expression vector carrying the OXHS4 gene of the present invention can be introduced into plant cells with Ti plasmid or plant viral vector using the conventional biological technology methods such as direct DNA transformation, microinjection and electroporation (Weissbach, 1998, Method for Plant Molecular Biology VIII, Academy Press, New York, pp. 411-463; Geiserson and Corey, 1998, Plant Molecular Biology (2nd Edition)).

The expression carriers comprising the OXHS4 gene of the present invention can be transformed into multiple hosts including rice to cultivate the plant varieties with drought resistance.

### Plant Cell Transformation Methods

Numerous methods for transforming plant cells with recombinant DNA are known in the art and may be used in the present invention. Two commonly used methods for plant transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn) and U.S. Pat. No. 6,153,812 (wheat) and Agrobacterium-mediated transformation is described in U.S. Pat. No. 5,159,135 (cotton); U.S. Pat. No. 5,824,877 (soybean); U.S. Pat. No. 5,591,616 (corn); and U.S. Pat. No. 6,384,301 (soybean), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly, i.e. at a non-specific location, in the genome of a target plant line. In special cases it may be useful to target recombinant DNA insertion in order to achieve site-specific integration, for example to replace an existing gene in the genome, to use an existing promoter in the plant genome, or to insert a recombinant polynucleotide at a predetermined site known to be active for gene expression. Several site specific recombination systems exist which are known to function implants include cre-lox as disclosed in U.S. Pat. No. 4,959,317 and FLP-FRT as disclosed in U.S. Pat. No. 5,527,695.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, for example various media and recipient target cells, transformation of immature embryo cells and subsequent regeneration of fertile transgenic plants are disclosed in U.S. Pat. Nos. 6,194,636 and 6,232,526, which are incorporated herein by reference.

The seeds of transgenic plants can be harvested from fertile transgenic plants and be used to grow progeny generations of transformed plants of this invention including hybrid plants line for selection of plants having an enhanced trait. In addition to direct transformation of a plant with a recombinant DNA, transgenic plants can be prepared by crossing a first plant having a recombinant DNA with a second plant lacking the DNA. For example, recombinant DNA can be introduced into first plant line that is amenable to transformation to produce a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example drough resistance or pest resistance, to produce progeny plants having recombinant DNA that confers both traits.

In the practice of transformation DNA is typically introduced into only a small percentage of target plant cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptll), hygromycin B (aph IV) and gentamycin (aac3 and aacCA) or resistance to herbicides such as glufosinate {bar or pat) and glyphosate (aroA or EPSPS). Examples of such selectable are illustrated in U.S. Pat. Nos. 5,550,318; 5,633,435; 5,780,708 and 6,118,047. Selectable markers which provide an ability to visually identify transformants can also be employed, for example, a gene expressing a colored or fluorescent protein such as a luciferase or green fluorescent protein (GFP) or a gene expressing a fteto-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO2, and 25-250 microeinsteins m⁻¹s⁻¹ of light, prior to transfer to a greenhouse or growth chamber for maturation. Plants are regenerated from about 6 weeks to 10 months after a transformant is identified, depending on the initial tissue. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

### Transgenic Plants and Seeds

Transgenic plants derived from the plant cells of this invention are grown to generate transgenic plants having an enhanced trait as compared to a control plant and produce transgenic seed and haploid pollen of this invention. Such plants with enhanced traits are identified by selection of transformed plants or progeny seed for the enhanced trait. For efficiency a selection method is designed to evaluate multiple transgenic plants (events) including the recombinant DNA, for example multiple plants from 2 to 20 or more transgenic events. Transgenic plants grown from transgenic seed provided herein demonstrate improved agronomic traits that contribute to increased yield or enhanced water deficit tolerance or both.

Not all transgenic events will be in transgenic plant cells that provide plants and seeds with an enhanced or desired trait depending on factors, such as location and integrity of the recombinant DNA, copy number, unintended insertion of other DNA, etc. As a result transgenic plant cells of this invention are identified by screening transformed progeny plants for enhanced water deficit stress tolerance and yield. For efficiency a screening program is designed to evaluate multiple transgenic plants preferably with a single copy of the recombinant DNA from 2 or more transgenic events.

Since the expression of the gene of the present invention is induced by drought stress, the gene of the present invention can be inserted into suitable expression carrier with combination with any drought inducible promoter of interest and transformed into plant hosts, wherein the gene expression thereby can be induced by drought condition, enhancing the drought resistance of the plant thereof.

The further illustration of the present invention is given below in reference to the figures and examples.

### BRIEF DESCRIPTION OF THE FIGURES

SEQ ID NO: 1 is the nucleotide acid sequence containing the coding region of the OXHS4 gene isolated and cloned in the present invention and the amino acid sequence of the corresponding protein thereof is set forth in SEQ ID NO: 2 with 628 amino acids in length.

FIG. 1. The expression pattern of OXHS4 gene under various adverse situations and hormone treatments. The samples are treated as follows: drought treatment for 0 h (CK), 0.5 h, 2 h, 4 h and 8 h; salt treatment for 0 h, 0.25 h, 0.5 h, 1 h and 2 h; cold treatment for 0 h, 0.5 h, 1 h, 3 h and 6 h; abscisic acid (ABA) treatment for 0 h, 0.5 h, 2 h, 4 h and 8 h.

FIG. 2. Identification of OXHS4 overexpression, mutant and complementary plant lines. A, Northern blot analysis of the OXHS4 overexpression plant (5 and 14 are the identified overexpression line and WT is wild type control); B, Determination of the insertion sites of T-DNA in the oxhs4 mutant (M5 and M8 are the identified homozygous lines); C, Detection of the expression level of OXHS4 gene; D-E, Detection of the positive (D) and the expression level (E) of the complementary plants (CP) (1-17 are 17 mutant complementary plants and WT and oxhs4 are detection controls).

FIG. 3. Phenotypes of the OXHS4 overexpression, mutant and complementary plant at seedling stage under drought stress. A-B, The growth of the transgenic plants before stress (A) and after rewatering (B); C, Survival statistics after stress; D, Measurement of free proline before and after stress, CK represents the proline content before stress; Dr represents the proline content after stress. Standard errors are based on triple biological repeats. (U5 and U14 are the overexpression lines, M5 and M8 are the homozygous mutant lines, CP is the positive complementary plant line and HY and ZH11 are the wild type controls for the mutant and overexpression.)

FIG. 4. Drought stress of the OXHS4 overexpression and mutant plants at adult plant stage and measurement of the root length and root volume. A and D are the growth states before drought stress and B, C, E and F are the growth states after drought stress. G-L, phenotypes of roots under normal growth condition and various drought stress levels. M-O, the relative root length and relative root volume under normal growth (M), medium drought stress (N) and serious drought stress (O). “*” and “**” mean P value of t-test is less than 0.05 and 0.01 respectively. (U5 and U14 are the overexpression lines, M5 and M8 are the homozygous mutant lines and HY and ZH11 are the wild type controls for the mutant and overexpression.)

FIG. 5. The relative yields (A) and relative fertilities (B) of the OXHS4 overexpression and mutant plants at adult plant stage under medium drought stress. ‘**’ means P value oft-test is less than 0.01. (OX is the overexpression line, oxhs4 is the homozygous mutant line and HY and ZH11 are the wild type controls of the mutant and overexpression.)

### EXAMPLES

The following examples are included to demonstrate embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments which are disclosed and still obtain a like or similar result without departing from the spirit and scope of the invention, therefore all matter set forth or shown in the accompanying drawings and examples is to be interpreted as illustrative and not in a limiting sense.

The following examples describe methods for isolating OXHS4 T-DNA insertion mutant and cloning the DNA fragment comprising the entire encoding region of OXHS4 gene and for verifying the function of OXHS4 gene.

### Example 1

### Determination of the Expression Level of the Endogenous Rice OXHS4 Gene Induced by Stress

To preliminarily judge whether the OXHS4 gene is related to stress response, the applicant firstly determined whether the expression level of the endogenous rice OXHS4 gene is induced under stress conditions. The applicant selected indica rice Minghui 63 (Oryza sativa L. ssp. Indica, from Fujian Academy of Agriculture Sciences) as the line for analyzing the expression pattern. The 4-leaf seedlings originating from sprouting seeds under normal growth conditions were treated under various stress conditions or hormones. The drought treatment was conducted by directly exposing the seedlings in air to lose water for 0 h, 0.5 h, 2 h, 6 h, and then sampling. The high-salinity treatment was conducted by transferring the seedlings into 200 mmol/L NaCl aqueous solution for 0 min, 15 min, 30 min, 1 h, 2 h, and then sampling. The coldness treatment was conducted by placing the seedlings in a 4° C. growth chamber for 0 h, 0.5 h, 1 h, 6 h, and then sampling. Hormone treatment was conducted by evenly spraying abscisic acid (ABA) to the surface of the plants and sampling at specific time points. The whole treatment and sampling process was conducted under continuous lighting condition. Total rice RNA was extracted using TRIZOL reagent (from Invitrogen Co.) according to the specification of the manufacturer and reverse transcribed into cDNA using reverse transcriptase SSIII (from Invitrogen Co.) according to the specification of the manufacturer. The reaction was conducted as follows: 65° C. 5 min, 50° C. 120 min, 70° C. 10 min. With the above reverse transcribed cDNA as template, OXHS4 gene was specifically PCR amplified using primers (OXHS4-1F: 5′-TGACGAGGTCTACAAGGCCGT-3′ and OXHS4-1R: 5′-ACACCACGTAGCTGCCGCT-3′). Meanwhile, a 76 bp fragment of the rice Actin/gene (Accession No. X16280) was specifically amplified with primers (AF: 5′-TGGCATCTCTCAGCACATTCC-3′ and AR: 5′-TGCACAAT GGATGGGTCAGA-3′) as internal control for quantitative analysis. PCR reaction was conducted as follows: 95° C. 10 sec; 95° C. 5 sec, 60° C. 34 sec, 40 cycles. Fluorescent real time quantitative analysis was conducted during the reaction process. The results (FIG. 1) demonstrated that the expression level of OXHS4 gene (SEQ NO:1) was enhanced upon induction by drought stress, while exhibited no significant change when treated under other conditions.

### Example 2

### Construction and Genetic Transformation of the Overexpression Vector of OXHS4 Gene

In order to verify the stress resistant function of OXHS4 gene, it was overexpressed in rice to investigate the gene function by the phenotype of transgenic plants thereof. The overexpression vector was constructed as follows: firstly, by searching in two databases, the Annotation No. of OXHS4 gene is LOC_Os02g19130 and AK242745 in RGAP(http://rice.plantbiology.msu.edu/) and KOME (http://cdna01.dna.affrc.go.jp/cDNA/) respectively, whose complete nucleotide sequence is set forth in SEQ ID NO:1 and is 2215 bp in length. The coding region of said gene is 1887 bp in length and the corresponding amino acid sequence is 628 amino acids in length, and based on which the primers were designed. With cDNA of the young panicle of indica rice Minghui 63 as template, using primers OXHS4OXF (5′-AGGAGGCGCTTCAGGTCT-3′, sequence specific primer with one KpnI cleavage site) and OXHS4OXR (5′-GCATAATCTTCCAGTTTCAG-3′, sequence specific primer with one BamHI cleavage site), a cDNA fragment comprising the complete coding region of OXHS4 gene was amplified, which corresponds to the 42-2063 bp of the sequence of the present invention. PCR reaction was conducted as follows: predenaturation at 94° C. for 3 min; 94° C. for 40 sec, 55° C. for 40 sec, 72° C. for 2 min, 32 cycles; and elongation at 72° C. for 5 min. The PCR product obtained by amplification was linked into pGEM-T vector (from Promega Co.) and the positive clone was screened and sequenced, resulting in the desired full length gene. Then, enzymatically cleave the positive clone plasmid with KpnI+BamHI, and recover exogenous fragments; meanwhile, enzymatically cleave the genetic transformation vector pCAMBIA1301U with the ubiquitin promoter in the same way (pCAMBIA1301U was reconstructed based on genetic transformation vector pCAMBIA1301 common used internationally, an Agrobacterium mediated vegetable genetic transformation vector carrying corn ubiquitin promoter with constitutive and overexpression characteristics); after cleavage, extract and purify the products of enzymatical cleavage with chloroform: iso-pentanol (24:1 v/v). Conduct linkage reaction between the digested OXHS4 fragment and digested pCAMBIA1301U vector and then transform the same into E. coli DH10β (the E. coli DH10β strain was purchased from Promega Co.). Identify the positive clone by enzymatic cleavage and the obtained recombination vector was named as OXHS4-OX-p1301U (wherein the nucleotide sequence of OXHS4 gene is set forth in SEQ ID NO:1 with 2215 bp in length, wherein position 63-1949 corresponds to the coding region with 1887 bp in length).

By using the Agrobacterium mediated rice genetic transformation method (described as follows), the above overexpression vector OXHS4-OX-p1301U was introduced into the rice variety “Zhonghua 11” (from Crop Research Institute, Chinese Agriculture Academy), and transgenic plant strains was then obtained by precultivation, infestation, co-culture, screening the callus with hygromycin resistance, differentiation, rooting, seedling training and transplanting. The above Agrobacterium mediated rice (Zhonghua 11) gentetic transformation method (system) was optimized based on the method reported by Hiei, et al (Hiei, et al., Efficient transformation of rice, Oryza saliva L., mediated by Agrobacterium and sequence analysis of the boundaries of the T-DNA, Plant J, 6:271-282,1994).

### The Particular Genetic Transformation Procedure of the Example is Following:

### (1) Reagents and Solutions

The abbreviations of the plant hormones used in the mediums of the present invention are: 6-BA (6-benzyladenine); CN (Carbenicillin); KT (Kinetin); NAA (naphthylacetic acid); IAA (indoleacetic acid); 2,4-D (2,4-dichlorphenoxyacetic acid); AS (Acetosyringone); CH (casein hydrolysate); HN (hygromycin); Dimethyl sulfoxide (DMSO); N6max (N6 solution with major elements); N6mix (N6 solution with trace elements); MSmax (MS solution with major elements); MSmix (MS solution with trace elements).

### (2) The Formulations of the Main Solutions

1) Preparation of Concentrated Solution of N6 Medium with Major Elements (10× Concentrated Solution):

| | |
|---|---|
| Potassium nitrate (KNO₃) | 28.3 g |
| Monopotassium phosphate (KH₂PO₄) | 4.0 g |
| Ammonium sulfate ((NH₄)₂SO₄) | 4.63 g |
| Magnesium sulfate (MgSO₄•7H₂O) | 1.85 g |
| Calcium chloride (CaCl₂•2H₂O) | 1.66 g |

Dissolve them one by one and add water to the final volume 1000 ml at room temperature.
2) Preparation of Concentrated Solution of N6 Medium with Trace Elements (10× Concentrated Solution):

| | |
|---|---|
| Potassium iodide (KI) | 0.08 g |
| Boracic acid (H₃BO₃) | 0.16 g |
| Manganese sulfate (MnSO₄•4H₂O) | 0.44 g |
| Zinc sulfate (ZnSO₄•7H₂O) | 0.15 g |

Dissolve and a3) Preparation of the ferric salt (Fe₂EDTA) stock solution (100×) 800 ml of double distilled water was heated to 70° C., 3.73 g of Disodium Ethylene Diamine Tetraacetic Acid (Na₂EDTA.2H2O) was added therein. After dissolving completely, the solution was kept in 70° C. water bath for 2 hours and water was added to a final volume 1000 ml and then the solution was kept at 4° C. for use.

### 4) Preparation of the Vitamin Stock Solution (100×)

dd water to the final volume 1000 ml at room temperature.

7) Preparation of the 2,4-D Stock Solution (1 mg/Ml)

Weigh 100 mg 2,4-D, dissolve in 1 ml 1 N potassium hydroxide for 5 min, then add 10 ml distilled water to dissolve followed by adding water to a final volume 100 ml and keep the solution at room temperature.

8) Preparation of the 6-BA Stock Solution (1 mg/Ml)

Weigh 100 mg 6-BA, dissolve in 1 ml 1 N potassium hydroxide for 5 min, then add 10 ml distilled water to dissolve followed by adding water to a final volume 100 ml and keep the solution at room temperature.

9) Preparation of the Naphthylacetic Acid (NAA) Stock Solution (1 mg/Ml):

Weigh 100 mg NAA, dissolve in 1 ml 1 N potassium hydroxide for 5 min, then add 10 ml distilled water to dissolve completely followed by adding water to a final volume 100 ml and keep the solution at 4° C. for use.

10) Preparation of the Indoleacetic Acid (IAA) Stock Solution (1 mg/Ml):

Weigh 100 mg IAA, dissolve in 1 ml 1 N potassium hydroxide for 5 min, then add 10 ml distilled water to dissolve completely followed by adding water to a final volume 100 ml and keep the solution at 4° C. for use. Add 300 ml distilled water and 2.78 g ferric sulfate (FeSO₄.7H₂O) into a large triangular flask. Add 300 ml distilled water into another large triangular flask.

11) Preparation of the Glucose Stock Solution (0.5 g/Ml):

Weigh 125 g glucose, then dissolve with distilled water by adding water to a final volume 250 ml followed by sterilization and store the solution at 4° C.

### 12) Preparation of the AS Stock Solution:

Weigh 0.392 g AS, dissolve completely in 10 ml DMSO, distribute into 1.5 ml centrifuge tubes and store at 4° C. for use.

### 13) 1N Potassium Hydroxide Stock Solution

To weigh 5.6 g potassium hydroxide, then dissolve with distilled water by adding water to a final volume 100 ml and store the solution at 4° C. for use.

### (3) The Formulations of the Mediums for Rice Genetic Transformation

### 1) Induction Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 2,4-D stock solution | 2.5 | ml |
| Proline | 0.3 | g |
| CH | 0.6 | g |
| Sucrose | 30 | g |
| Phytagel | 3 | g |

Add distilled water to 900 ml, adjust pH to 5.9 with 1N potassium hydroxide, boil the solution, add water to a final volume 1000 ml and distribute the solution into 50 ml-triangular flasks (25 ml/flask) followed by seal and sterilization.

### 2) Subculture Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 2,4-D stock solution | 2.0 | ml |
| Proline | 0.5 | g |
| CH | 0.6 | g |
| Sucrose | 30 | g |
| Phytagel | 3 | g |

Add distilled water to 900 ml, adjust pH to 5.9 with 1 N potassium hydroxide, boil the solution, add water to a final volume 1000 ml and distribute the solution into 50 ml-triangular flasks (25 ml/flask) followed by seal and sterilization.

### 3) Preculture Medium

| | | |
|---|---|---|
| N6max concentrated solution (10X) | 12.5 | ml |
| N6mix concentrated solution (100X) | 1.25 | ml |
| Fe²⁺EDTA stock solution (100X) | 2.5 | ml |
| Vitamin stock solution (100X) | 2.5 | ml |
| 2,4-D stock solution | 0.75 | ml |
| CH | 0.15 | g |
| Sucrose | 5 | g |
| Agar powder | 1.75 | g |

Add distilled water to 250 ml, adjust pH to 5.6 with 1 N potassium hydroxide followed by seal and sterilization. Prior to use, heat and dissolve the medium, add 5 ml glucose stock solution and 250 μl AS stock solution and distribute the solution into dishes (25 ml/dish).

### 4) Coculture Medium

| | | |
|---|---|---|
| N6max concentrated solution (10X) | 12.5 | ml |
| N6mix concentrated solution (100X) | 1.25 | ml |
| Fe²⁺EDTA stock solution (100X) | 2.5 | ml |
| Vitamin stock solution (100X) | 2.5 | ml |
| 2,4-D stock solution | 0.75 | ml |
| CH | 0.2 | g |
| Sucrose | 5 | g |
| Agar powder | 1.75 | g |

Add distilled water to 250 ml, adjust pH to 5.6 with 1 N potassium hydroxide followed by seal and sterilization. Prior to use, heat and dissolve the medium, add 5 ml glucose stock solution and 250 μl AS stock solution and distribute the solution into dishes (25 ml/dish).

### 5) Suspension Culture Medium

| | | |
|---|---|---|
| N6max concentrated solution (10X) | 5 | ml |
| N6mix concentrated solution (100X) | 0.5 | ml |
| Fe²⁺EDTA stock solution (100X) | 0.5 | ml |
| Vitamin stock solution (100X) | 1 | ml |
| 2,4-D stock solution | 0.2 | ml |
| CH | 0.08 | g |
| Sucrose | 2 | g |

Add distilled water to 100 ml, adjust pH to 5.4 and distribute the solution into two 100 ml-triangular flasks followed by sealing and sterilization. Prior to use, add 1 ml glucose stock solution and 100 μl AS stock solution.

### 6) Selection Culture Medium

| | | |
|---|---|---|
| N6max concentrated solution (10X) | 25 | ml |
| N6mix concentrated solution (100X) | 2.5 | ml |
| Fe²⁺EDTA stock solution (100X) | 2.5 | ml |
| Vitamin stock solution (100X) | 2.5 | ml |
| 2,4-D stock solution | 0.625 | ml |
| CH | 0.15 | g |
| Sucrose | 7.5 | g |
| Agar powder | 1.75 | g |

Add distilled water to 250 ml and adjust pH to 6.0 followed by sealing and sterilization. Prior to use, dissolve the medium, add 250 μl HN and 400 ppm CN and distribute the medium into dishes (25 ml/dish).

### 7) Predifferentiation Medium

| | | |
|---|---|---|
| N6max concentrated solution (10X) | 25 | ml |
| N6mix concentrated solution (100X) | 2.5 | ml |
| Fe²⁺EDTA stock solution (100X) | 2.5 | ml |
| Vitamin stock solution (100X) | 2.5 | ml |
| 6-BA stock solution | 0.5 | ml |
| KT stock solution | 0.5 | ml |
| NAA stock solution | 50 | μl |
| IAA stock solution | 50 | μl |
| CH | 0.15 | g |
| Sucrose | 7.5 | g |
| Agar powder | 1.75 | g |

Add distilled water to 250 ml, adjust pH to 5.9 with 1N potassium hydroxide followed by seal and sterilization. Prior to use, dissolve the medium, add 250 μl HN and 200 ppm CN and distribute the medium into dishes (25 ml/dish).

### 8) Differentiation Medium

| | | |
|---|---|---|
| N6max concentrated solution (10X) | 100 | ml |
| N6mix concentrated solution (100X) | 10 | ml |
| Fe²⁺EDTA stock solution (100X) | 10 | ml |
| Vitamin stock solution (100X) | 10 | ml |
| 6-BA stock solution | 2 | ml |
| KT stock solution | 2 | ml |
| NAA stock solution | 0.2 | ml |
| IAA stock solution | 0.2 | ml |
| CH | 1 | g |
| Sucrose | 30 | g |
| Phytagel | 3 | g |

Add distilled water to 900 ml, adjust pH to 6.0 with 1 N potassium hydroxide, boil the solution, add water to a final volume 1000 ml and distribute the solution into 50 ml-triangular flasks (50 ml/flask) followed by seal and sterilization.

### 9) Rooting Medium

| | | |
|---|---|---|
| MSmax concentrated solution (10X) | 50 | ml |
| MSmix concentrated solution (100X) | 5 | ml |
| Fe²⁺EDTA stock solution (100X) | 5 | ml |
| Vitamin stock solution (100X) | 5 | ml |
| Sucrose | 30 | g |
| Phytagel | 3 | g |

Add distilled water to 900 ml, adjust pH to 5.8 with 1 N potassium hydroxide, boil the solution, add water to a final volume 1000 ml and distribute the medium into rooting tubes (25 ml/flask) followed by seal and sterilization.

### (4) The Agrobacterium Mediated Genetic Transformation Protocol

### 4.1 Callus Induction:

(1) Mature rice seeds were deshelled, then treated with 70% alcohol for 1 minute and disinfected on the surface of the seeds with 0.15% HgCl₂ for 15 minutes;
(2) The seeds were washed with sterilized water for 4-5 times;
(3) The sterilized seeds were put on the induction medium;
(4) The inoculated callus induction medium was placed in darkness and cultured for 4 weeks at 25±1° C.

### 4.2 Callus Subculture:

The bright yellow, compact and relatively dry embryogenic callus was selected, put onto the subculture medium, and cultured in darkness for 2 weeks at 25±1° C.

### 4.3 Pre-Culture:

The compact and relatively dry rice embryogenic callus was selected, put onto the pre-culture medium, and cultured in darkness for 2 weeks at 251° C.

### 4.4 Agrobacterium Culture:

Agrobacterium EHA105 (kind gift from Prof. Lin Yongjun of State Key Laboratory of Crop Genetic Improvement, Huazhong Agricultural University) was precultured on the selective LA medium at the temperature of 28° C. for 2 days;

Said Agrobacterium was transferred into suspension medium in swing bed at 28° C. for 2-3 hours.

### 4.5 Agrobacterium Infection:

(1) The pre-cultured callus was transferred into a sterilized bottle;
(2) The Agrobacterium suspension was adjusted to OD₆₀₀0.8-1.0;
(3) The callus was immersed in the Agrobacterium suspension for 30 minute;
(4) The callus was transferred on sterilized filter paper and dried; and then cultured onto the cocultivation medium for 2 days at 19-20° C.

### 4.6 Washing and Selection Culture of Callus:

(1) The callus was washed with sterilized water until no agrobacrium was observed;
(2) The callus was immersed in sterilized water containing 400 ppm carbenicillin (CN) for 30 minutes;
(3) The callus was transferred on sterilized filter paper and dried;
(4) The callus was transferred on the selection medium and cultured and selected for 2-3 times, 2 weeks for each time (The hygromycin concentration was 400 ppm for the first culture, and 250 ppm for the second and subsequent cultures)

### 4.7 Differentiation:

The resistant calluses were transferred to the pre-differentiation medium and cultured in darkness for 5-7 weeks;

The pre-differentiated calluses were then transferred to differentiation culture medium and cultured in lighting at 26° C.

### 4.8 Rooting:

(1) The roots generated during the differentiation were cut off;
(2) Then the plant was transferred to the rooting culture medium and cultured in lighting at 26° C. for 2-3 weeks.

### 4.9 Transplantation:

The residual medium on roots of the plant was washed off, the seedlings with well-grown roots were transplanted into fields for growing until being harvested.

### Example 3

### Drought Stress Experiments of OXHS4 Transgenic Overexpression Rice

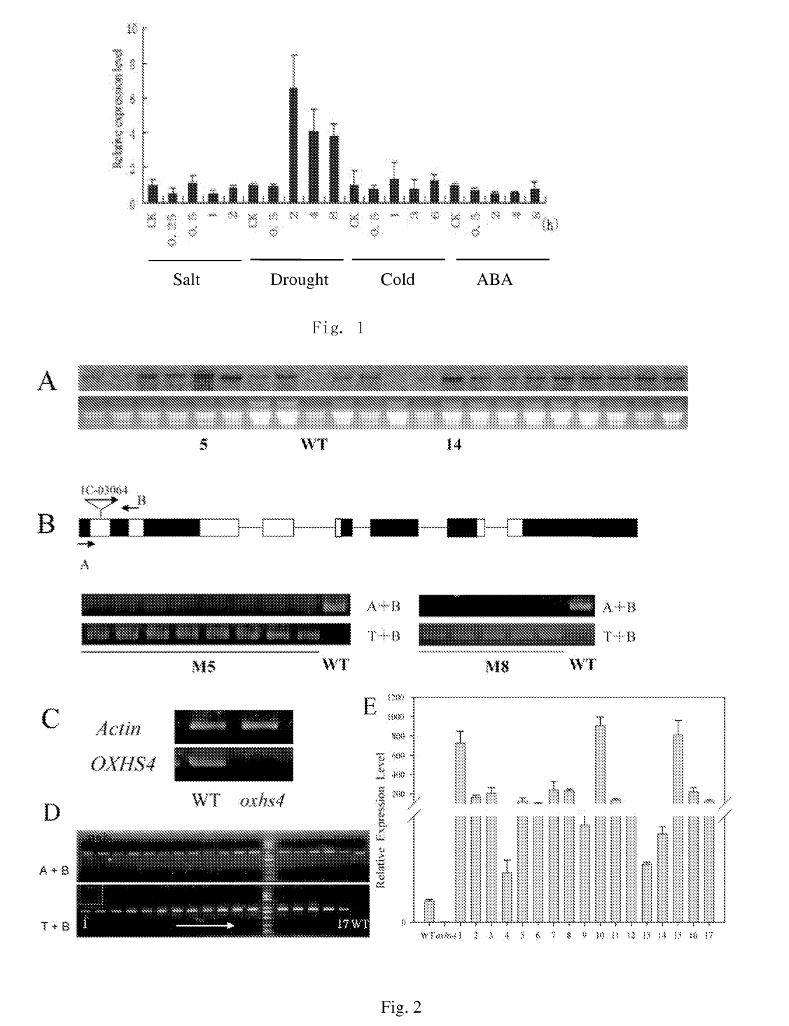

In the present invention, the expression of OXHS4 gene in the transgenic rice plants obtained in the above Example 2 was detected by Northern hybridization method. Total RNAs of the leaves were extracted (Trizol reagent, purchased from Invitrogen Co.) followed by transferring to membrane according to the protocols recited in Molecular Cloning (Science Press, 1999) and subjected to Northern hybridization with OXHS4 as probe. The results of the expression amount determination (FIG. 2A) show that the expression amount of OXHS4 gene in most transgenic plants is significantly enhanced relative to that in wild type (Zhonghua 11).

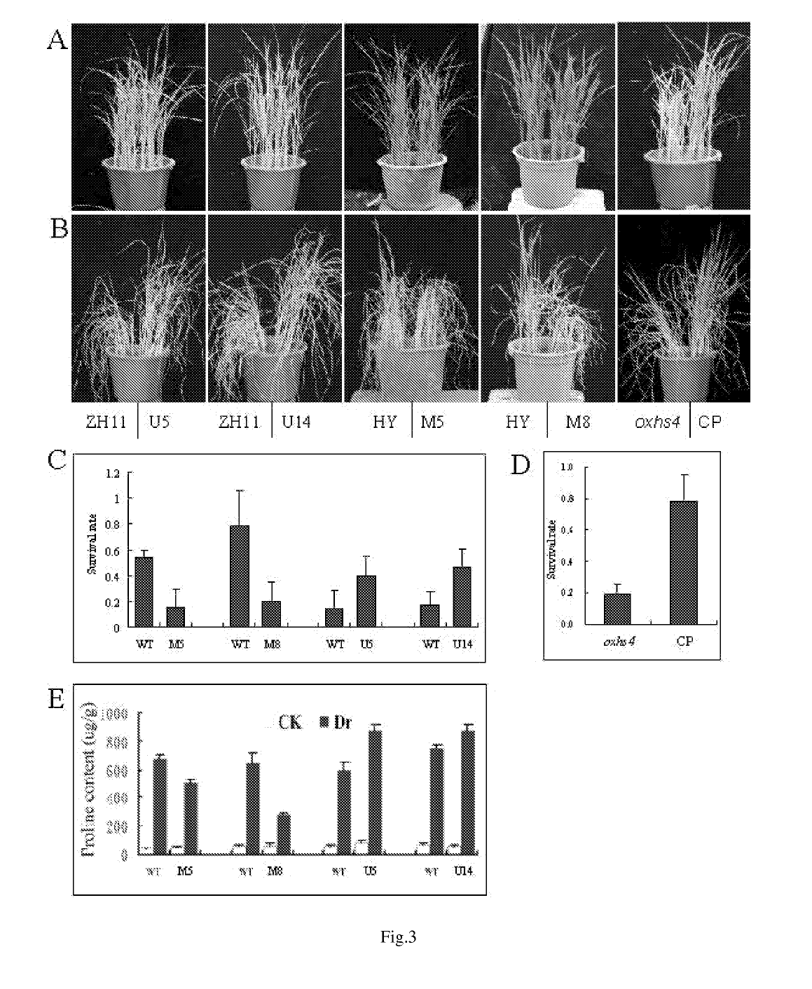

For verifying this deduction, the applicant conducted the drought stress experiments with OXHS4 overexpression plant at seedling stage. The seeds of two overexpression lines (U5 and U14) and the corresponding wild type plant Zhonghua 11 were germinated in ½ MS medium (The seeds of the two overexpression lines were germinated in medium in the presence of 50 mg/L hygromycin to exclude negative transgenic plants), the plants germinating for one week were planted in small red buckets with equivalent soil with 20 transgenic plants and 20 wild type plants (Zhonghua 11) in each bucket respectively and the plants in each bucket were stopped water supply at 3 to 4 leaves stage (FIG. 3A) until leaves completely rolled and leaf apex became white and then rewatered. The survival rates of transgenic lines and the corresponding wild type plants were observed. It can be found from FIG. 3B that after 7 days of rewatering, almost all wild type control plants Zhonghua 11 withered, while part of the plants in overexpression line (transgenic plants of the present invention) were still alive. The further survival statistics (FIG. 3C) shows that the survival rate in wild type Zhonghua 11 is less than 20%, while the survival rate of transgenic overexpression plants is about 50%, remarkably higher (P value of t-test is less than 0.05) than that of wild type plants in the same bucket. Upon environment stress, the proline content of plants is remarkably increased. The proline content reflects stress resistance of plants to some extent and more proline generally accumulates in stronger stress resistant plants. Therefore, proline content can be measured as an important physiological index reflecting stress resistance of plants. The result of proline content measurement was consistent with that of drought stress, i.e. the proline contents in all plants were substantially at the same level before stress, while the increase of proline content in overexpression plants was remarkably higher than that in wild type plants (P value oft-test is less than 0.05) (FIG. 3E). The results of drought stress experiment show that OXHS4 transgenic overexpression plants have stronger drought resistance than wild type plants at seedling stage.

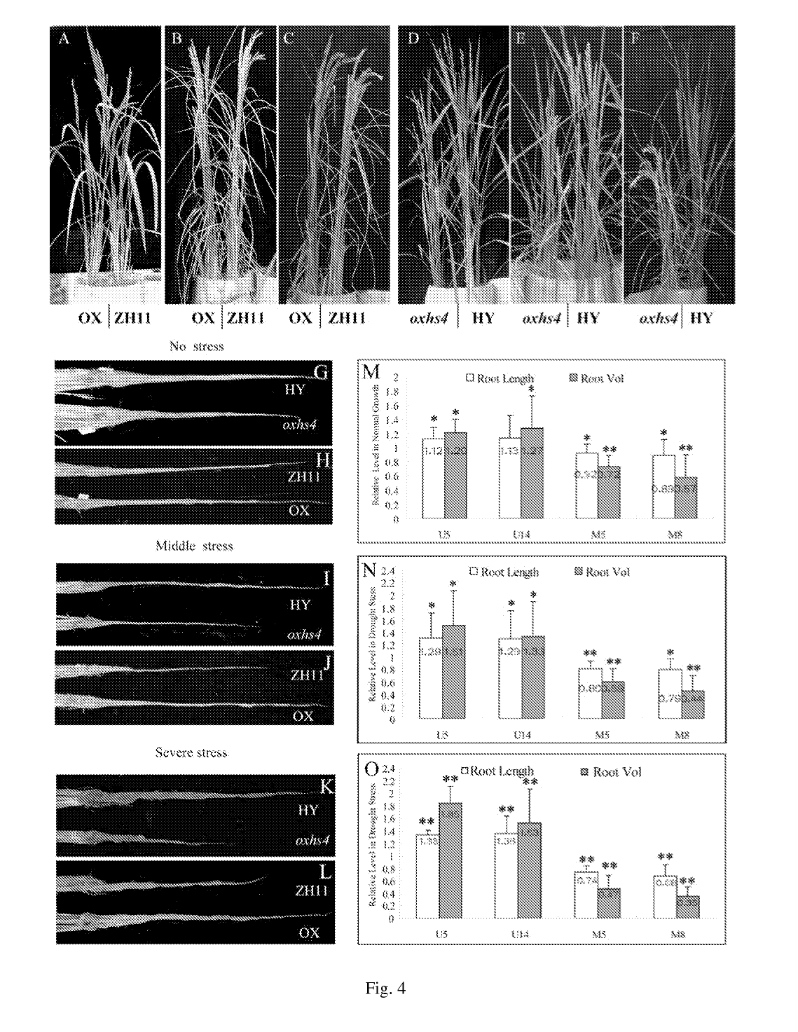

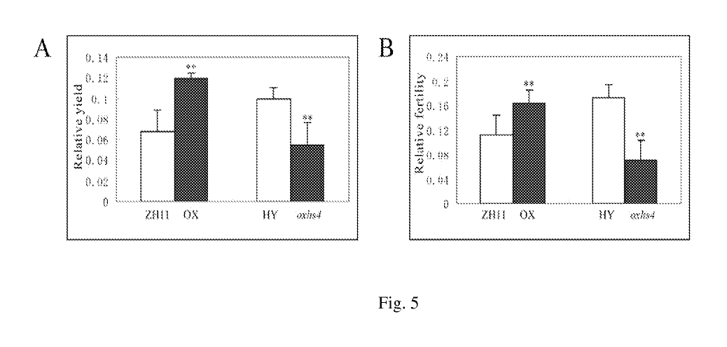

OXHS4 overexpression lines (U5 and U14) were treated with different agrees of drought stress at adult plant stages in PVC tubes. The overexpression plants were planted in PVC tubes and meanwhile the corresponding wild type plants (Zhonghua 11) were planted in PVC tubes as control, with 4 plants in each line. The experiment was in triplet. When young panicle develops to meiosis stage of pollen mother cell (i.e. young panicle is 5-10 cm in length) during rice adult plant stage, which is the key period determining whether spikelet can completely develop and the size of grain, it is most sensitive to the environment conditions and the shortage of water at this stage results in spikelet degeneration, heading delay and reduced seed setting. Given it will take a period of time for plants to sense drought stress, we stopped water supply for 15-20 days early at meiosis stage of pollen mother cell (depending on the weather, covered with removable canopy in case of rain), and then rewatered. Under normal growth conditions, the agronomic traits of overexpression plants exhibit no difference from that of wild type Zhonghua 11 in the buckets as well as in PVC tubes (FIG. 4A). However, when being treated by severe drought stress followed by rewatering, the applicant found that the control Zhonghua 11 (ZH11) withered and became yellow, while overexpression plants were still green and retained growth activity (FIGS. 4B and C). The yield of single plant and seed setting rate of OXHS4 overexpression plants under normal condition and medium drought stress were measured (Since under severe drought stress, plants are hard to produce seeds, the yield and seed setting were not measured). Owing to environmental effect, we employed relative yield (yield of single plant under stress condition/yield of single plant under normal condition) and relative seed setting rate (seed setting rate of plant under stress condition/seed setting rate of plant under normal condition) as comparative indices. The result shows that the relative yield (FIG. 5A) and relative seed setting rate (FIG. 5B) are remarkably higher than that of wild type ZH11. The above results demonstrate that overexpression of OXHS4 can increase drought resistance in rice.

In addition to the above agronomic traits, root length and root volume were also measured. Since the activity of OXHS4 promoter in root is significantly increased under osmosis stress, it is of great significance to exploit whether the drought resistant mechanism of OXHS4 is related to root growth. As shown in FIG. 4G-L, the roots of overexpression plants grew better than wild type ZH11 (FIG. 4H), and the difference became increasingly obvious under worsening drought stress, especially under severe drought stress (FIG. 4L). This is conformed by the statistics of the relative root length and relative root volume under normal growth (FIG. 4M), medium drought stress (FIG. 4N) and serious drought stress (FIG. 4O) (Relative level is referred to the ratio of root length or volume of transgenic plants and that of wild type plants in the same PVC tube, the corresponding ratios are indicated on the column in FIG. 4M-O). During the process of root growth, the elongation of root apex is dependent on extension of cells in elongation zone, which permits root apex advancing in depth direction and obtaining more water and nutrient to survive drought stress. The results demonstrate that OXHS4 may control root growth to regulate rice drought resistance.

### Example 4

### Isolation of OXHS4 Gene Mutant

For further verifying the drought resistance function of OXHS4 gene, the applicant conducted the drought stress experiment with OXHS4 mutant. The T-DNA insertion mutant 1C-03064 of OXHS4 gene was selected from Rice T-DNA Insertion Sequence Database (RISD) (The starting material of the present invention is mutant 1C-03064, the website: http://signal.salk.edu/cgi-bin/RiceGE, POSTECH Plant Functional Genomics Laboratory of Korea). The methods of constructing this mutant vector and genetic transformation are described in the related literatures (Jeong et al., Generation of a flanking sequence-tag database for activation-tagging lines in japonica rice. Plant J. 2006, 45:123-32.) and the present description does not provide more description in detail. In the above mentioned mutant database, the sequence (with 560 bp in length) flanking the OXHS4 T-DNA mutant 1C-03064 is set forth as follows:

ATCAACTCACCTGGTACCTGGTACCTCGGATCCGTGACCTCAAGCTCTGG
GAANCCAGGTGGATGAGTCGTAGGCAATGAAAAGTTCANATGCCCATTCT
GTCATGGGTGAAGTAAGAATGCAGGACTACCGTTNCAACNAGCTACTTCA
NCATGCCATTGGGGTATGGCGCATCCAATCGCTCTCCAAAGGTGAAGGCA
NACCANNTGGNCTTGGCCANTCTTCTCAAGAACNACTATGCTGATGCANC
AGGCTCATTGCCATCACGACAGGCCATTGGACCAAGTAATCCTCCAAGGC
CATTGCAAGATCAGGAAGCGTATGTTTGGCCATGGATGGGCATCCTTGCA
AATGTTCCAGCTGAGAAAACAAAGGAGGATGGANCTAGTCTGATGCANCA
GCTANCTAATTTCAATCCCTTGCAGTTTACTGCTGCGCTCTGCTCCCAGG
TAGGTATACTGGTTATGCAGTTGTCCGTTTCNCANANATNGGATTGGGTT
CACGAACNCCTTGNNNTTNCANAACTNCTTNAAATCNNACGTCTGGNNAN
AAAGATTGCC
(Note: N means undetermined nucleotide
in sequencing).

According to the insertion site of T-DNA, primers flanking the insertion site (A:5′-TGAACACACATCAGTGAGTT-3′ and B:5 ′-CACCAAATACTTGCTCTTAG-3′) and at the boundary of vector (T:5′-TTGGGGTTTCTACAGGACGTAAC-3′) were designed. By detecting the insertion site with PCR, the result demonstrated that the insertion site is positioned at 100 bp downstream of the ATG site (see FIG. 2B) and two homozygous lines (M5 and M8) were isolated. The expression amount of OXHS4 gene was measured using RT-PCR and the result demonstrated that the expression of OXHS4 gene was completely inhibited (FIG. 2C). The primers used are OXHS4RTF:5′-CCGTGTCTGGTCTGGAAAA-3′ and OXHS4RTR:5′ TCTTGAGTCCGATGGTGCT-3′ and meanwhile, Actin was used as internal control for quantitative analysis.

### Example 5

### Identification of the Drought Stress Phenotype of the Mutant

The sprouting seeds of M5 and M8 homozygous oxhs4 mutant and wild type line Hwayoung (purchased from POSTECH Plant Functional Genomics Laboratory of Korea) were seeded in small round buckets. The soil used in the experiments was a mixture of South China rice soil and sands in a ratio of 2:3, the same amount of homologous soil with the same volume of water was added in each bucket and the water naturally leaked out to ensure the consistency of soil compactness. The experiment was in triplet. The healthy plants in 4-leaf stage were treated with drought stress for 6-10 days (depending on the particular weather), then rewatered for 5-7 days. Compared with the wild type control, T-DNA homozygous plants showed the drought susceptible phenotype (FIG. 3B). After rewatering, the survival rates of the homozygous lines are lower than 30%, while the survival rates of the wild type lines are higher than 60% (FIG. 3C). Upon drought stress, the increase of proline content in homozygous mutant plant is significantly (P value oft-test is lower than 0.05) lower that that in wild type plant (FIG. 3E). To exploit whether the pheonotypes are the result of OXHS4 deletion, other insertion or gain of function, the applicant introduced the overexpressed OXHS4 into homozygous oxhs4 callus to detect whether the above mentioned pheonotypes can be rescued. Full length OXHS4 was linked into pCAMBIA1301U, then introduced into the callus derived from homozygous oxhs4 seed and totally 17 transgenic complementary plants (CP) were obtained. By detection with primers A, B and T in Example 4, it was found that all the 17 transgenic plants were OXHS4 positive (FIG. 2D), suggesting that full length OXHS4 had been introduced into homozygous oxsh4 plants. The expression amounts of OXHS4 in young panicle of 1-17 complementary plants were measured using Real-Time PCR, with wild type Hwayoung (abbreviated as HY) and homozygous oxhs4 mutant as control and the primers used are OXHS4QF: 5′-GTGACCCAGTTGGTAGGTTTTTG-3′ and OXHS4QR:5′-TTGCGAGACAGGTCATTTTCC-3′). The results shows that the expression amounts of OXHS4 in the complementary plants are significantly higher than that in HY and oxhs4 mutant (FIG. 2E), suggesting that the introduced full length exogenous OXHS4 was expressed in oxhs4 mutant. The drought stress experiment at seedling stage shows that the complementary plants exhibited the drought resistant phenotype (FIG. 3B) and significantly enhanced survival rate (FIG. 3D) compared with oxhs4 mutant. These results demonstrate that OXHS4 deletion results in the decreased drought resistance in rice. Meanwhile, the applicant also conducted the drought stress experiment on oxhs4 mutant at adult plant stage in PVC tubes, as described in Example 3. During the process of medium drought stress, the leaves of homozygous mutant plants completely rolled, while the leaves of wild type plant HY still maintained unfolded, indicating better growth state compared with oxhs4 mutant plants. Upon rewatering after severe drought stress, as shown in FIG. 4F, wild type HY plants recovered rapidly and grew well, while oxhs4 mutant had withered (FIG. 4F). Measurement of root length and root volume demonstrates that under normal condition, the root of mutant plants grew worse than wild type HY (FIG. 4G), and the difference became increasingly obvious under worsening drought stress, especially under severe drought stress (FIGS. 4I and K). This is conformed by the statistics of the relative root length and relative root volume under normal growth (FIG. 4M), medium drought stress (FIG. 4N) and serious drought stress (FIG. 4O). Under medium drought stress, the relative yield (FIG. 5A) and relative seed setting rate (FIG. 5B) are remarkably lower than that of wild type HY. Therefore, loss of OXHS4 results in slower root growth rate, which becomes even more obvious under drought condition, thereby weakening the drought resistance of rice.

1-16. (canceled)

17. A DNA construct comprising a heterologous promoter operably linked to a polynucleotide encoding a XHS protein comprising the amino acid sequence as set forth in SEQ ID NO:2.

18. The DNA construct of claim 17, wherein said polynucleotide comprises the nucleotide sequence as set forth in SEQ ID NO:1 from nucleotide position 63 to nucleotide position 1949.

19. The DNA construct of claim 18, wherein said polynucleotide comprises the nucleotide sequence as set forth in SEQ ID NO:1.

20. The DNA construct of claim 17, wherein said promoter is a drought inducible promoter.

21. A transgenic plant or plant part comprising the DNA construct of claim 17, wherein said plant is drought tolerant and said plant part comprises seed or cell.

22. The transgenic plant or plant part of claim 21, wherein said plant is selected from the group consisting of a corn, cotton, soybean, rice and wheat plant.

23. A method of improving drought resistance of a plant, comprising providing a transgenic plant comprising the DNA construct of claim 17, wherein expression of said polynucleotide is induced under drought condition thereby improving the drought resistance of the transgenic plant.

24. The method of claim 23, wherein said providing step comprises transforming a plant with said DNA construct thereby obtaining said transgenic plant.

25. The method of claim 23, wherein said plant is selected from the group consisting of a corn, cotton, soybean, rice and wheat plant.

26. A DNA construct comprising a heterologous promoter operably linked to a polynucleotide encoding a XHS protein comprising the amino acid sequence as set forth in SEQ ID NO:2, wherein said promoter is an ubiquitin promoter.

27. The DNA construct of claim 26, wherein said promoter is a corn ubiquitin promoter.

28. The DNA construct of claim 26, wherein said polynucleotide comprises the nucleotide sequence as set forth in SEQ ID NO:1 from nucleotide position 63 to nucleotide position 1949.

29. The DNA construct of claim 28, wherein said polynucleotide comprises the nucleotide sequence as set forth in SEQ ID NO:1.

30. A transgenic plant or plant part comprising the DNA construct of claim 26, wherein said plant is drought tolerant and said plant part comprises seed or cell.

31. The transgenic plant or plant part of claim 30, wherein said plant is selected from the group consisting of a corn, cotton, soybean, rice and wheat plant.

32. A method of improving drought resistance of a plant, comprising providing a transgenic plant comprising the DNA construct of claim 26, wherein said polynucleotide is overexpressed compared to a wild type plant, said transgenic plant exhibiting improved drought resistance compared to the wild type plant.

33. The method of claim 32, wherein said providing step comprises transforming a plant with said DNA construct thereby obtaining said transgenic plant.

34. The method of claim 32, wherein said plant is selected from the group consisting of a corn, cotton, soybean, rice and wheat plant.

35. A method of sowing, planting, or growing a plant with improved drought resistance, said method comprising the step of sowing, planting, or growing a transgenic plant comprising the DNA construct of claim 17.

36. A method of sowing, planting, or growing a plant with improved drought resistance, said method comprising the step of sowing, planting, or growing a transgenic plant comprising the DNA construct of claim 26.
